SEQUENCE LISTING (6 sequences)

SEQ ID NO: 1 - protein, 7 aa, Artificial
  synthetic peptide
RRETAWA

SEQ ID NO: 2 - DNA, 22 bp, Artificial
  PCR primer
cagggaagagcgggcgctatgg

SEQ ID NO: 3 - DNA, 27 bp, Artificial
  PCR primer
gggagtctgaaattgggaggactcagg

SEQ ID NO: 4 - DNA, 40 bp, Artificial
  PCR primer
ggatccccgtgacttctttgccgtgaattcaagagattca

SEQ ID NO: 5 - DNA, 40 bp, Artificial
  PCR primer
aagcttaaaaagtgactactttgccgtgaatctcttgaat

SEQ ID NO: 6 - protein, 9 aa, Artificial
  synthetic peptide
CRRETAWAC

(19) United States
(12) Patent Application Publication
(10) Pub. No.: US 2011/0182916 A1
(43) Pub. Date: Jul. 28, 2011

(54) Use of Agonists of Integrin Alpha 5 for Inducing the Osteogenic Differentiation of Mesenchymal Stem Cells

(76) Inventors: Pierre Marie, Paris Cedex (FR); Fromigue Olivia, Paris Cedex (FR); Hamidouche Zahira, Le Kremlin Bicetre Cedex (FR)

(21) Appl. No.: 13/057,665

(22) PCT Filed: Aug. 5, 2009
(86) PCT No.: PCT/IB09/06826

§ 371 Date: Apr. 5, 2011

(30) Foreign Application Priority Data
Aug. 5, 2008 (EP) 08290752.8

(51) Int. Cl. A61K 39/395; C12N 5/0775; A61K 38/08; C07K 16/18; C07K 7/06; A61P 19/08

(52) U.S. Cl. 424/172.1; 435/366; 514/16.7; 530/389.1; 530/329

(57) ABSTRACT

The invention relates to the use of agonists of integrin alpha for promoting osteoblast differentiation of mesenchymal stem cells. These agonists are useful in particular for enhancing osteogenesis in the treatments of diseases associated with bone loss or insufficient bone formation.

The present invention relates to the use of agonists of integrin alpha 5 (ITGA5) for inducing the osteogenic differentiation of mesenchymal stem cells.

Bone healing in vivo is generally considered to be biologically optimal since the vast majority of defects in this tissue heal spontaneously with minimal treatment. However, in some cases healing is compromised because of either interposition of soft tissue, improper fracture fixation, loss of bone, metabolic disturbances, impairment of blood supply and infection. In addition, in certain clinical settings, large pieces of bone must be removed to treat benign and malignant tumours, osteomyelitis as well as bone deficiencies and abnormal loss in the maxillo-facial area.

In these challenging situations, autologous bone harvested from donor sites such as iliac crest is the preferred treatment. Grafts of this kind are osteoconductive (they provide a scaffold on which bone cells can proliferate), osteoinductive (they induce proliferation of undifferentiated cells and their differentiation into osteoblasts), and osteogenic (they provide a reservoir of skeletal stem and progenitor cells that can form new bone). Since the available autologous bone supplies are limited and harvesting autologous bone is painful and entails procedures with risk of infection, it has become necessary to develop alternative techniques to overcome these drawbacks. In the past, surgeons used banked bone and natural or synthetic substrates; such materials had limited success because they only provided a scaffold which had to be invaded by bone-forming bioactive cells.

For these reasons, several novel approaches are currently being explored, including the use of adult stem cells, as potential alternatives. Since, such “biological composites” will not depend on local recruitment of the osteocompetent cells needed for new bone synthesis, they will be of particular interest and useful in clinical cases in which the bed of the wound cannot provide these cells. Such cases include patients with large bone defects and those with reduced number of osteocompetent cells because of aging, osteoporosis, metabolic disturbances and irradiation treatment. Promising cell types currently under investigation for treatment of bone defects are mesenchymal stem cells (MSCs) derived from the bone marrow stroma.

These cells, in specific conditions, are able to differentiate in vitro and in vivo into osteogenic, adipogenic or chondrogenic lineage under appropriate environment.

In view of the potential of MSCs to differentiate in vitro into the osteogenic lineage, it has been proposed to use them as a source for bone tissue regeneration, and several studies have shown the ability of transplanted bone-marrow derived MSC to repair bone defects in vivo, through infusion or local implantation.

The osteogenic differentiation of MSCs is characterized by events characterized by cell proliferation, differentiation and production of an extracellular matrix (ECM), composed mainly of type I collagen and bone matrix proteins, which become progressively mineralized. It involves the expression of early and late genes that typify osteogenesis in vivo. Osteoblast commitment is characterized by the expression of Runx2, the main transcription factor required for osteoblast differentiation, which is weakly expressed in basal conditions in MSCs. Genes that are timely expressed during osteogenic differentiation of MSCs in vitro include alkaline phosphatase, type I collagen, osteopontin, bone sialoprotein and osteocalcin, the later being associated with the onset of mineralization.

Optimal osteogenic differentiation of MSCs is required for their efficient use for bone repair. Several factors have been shown to promote the osteogenic differentiation of MSCs in vitro (for review cf. for instance (MARIE & FROMIGUE, Regenerative Medicine, 1, 539-48, 2006); they include Bone Morphogenetic Proteins (BMPs), Wnt proteins, glucocorticoids such as dexamethasone, or extracellular matrix proteins (ECM). However, the effect of some of these factors appears to depend on the species from which the MSCs cultures are derived. For instance, dexamethasone mainly increases osteoblast differentiation of rat and human MSCs, whereas it also induces adipocyte formation in mouse-derived MSCs. Also, BMP are required for osteogenic differentiation of MSCs in some, but not all species.

Thus, although significant advances have been achieved in the knowledge of the mechanisms involved in osteogenic differentiation of MSCs, there is still a need of identifying factors that promote MSC differentiation towards functional osteogenic cells, in order to use them for treatment of bone defects in humans.

Integrins are a superfamily of cell-surface adhesion molecules formed from 18 different α chains (α₁-α₁₁, α_{V}, α_{IIb}, α_{L}, α_{M}, α_{X}, α_{D}, α_{E}) and eight different β chains (β₁-β₈) that assemble non-covalently as heterodimers. At the present time, more than 20 different αβ heterodimers have been described. Integrins play a major part in the mediation of cell-cell and cell-matrix interactions, and are implicated in major cellular functions such as cell growth, survival, differentiation, and migration. In osteoblasts, cell-matrix interactions mediated by some integrins are important modulators of cell differentiation (DAMSKY, Bone, 25, 95-6, 1999; FRANCESCHI, Crit. Rev Oral Biol Med, 10, 40-57, 1999; GLOBUS et al., ASGSB Bull, 8, 19-28, 1995; ZIMMERMAN et al., Dev Biol, 220, 2-15, 2000).

The integrin α5 subunit (ITGA5) is synthesised as a precursor of 1049 aa (Swissprot P08648), which after cleavage of a signal peptide of 41 aa, gives a mature protein of 1008 aa. This mature protein contains a site that is cleaved post-translationally to yield an N-terminal heavy chain of 853 aa, and a C-terminal light chain of 155 aa, linked by an interchain disulfide bond. Its N-terminal extracellular domain, of 951 residues, comprises a head containing a seven-bladed β-propeller structure, followed by a leg comprising three β-sandwich domains, termed “thigh”, “calf 1” and “calf 2”.

The α5 subunit combines with the β1 subunit to form the α5β1 integrin. This integrin belongs to the subgroup of RGD-binding integrins, which also includes integrins containing α_{IIb}, α₈ or α_{V} subunits. All these integrins are receptors for ligands containing Arg-Gly-Asp (RGD) motifs, such as fibronectin (FN), vitronectin (VN), fibrinogen, laminin, von Willebrand factor, or osteopontin.

The α5β1 integrin is mainly a cell surface receptor for fibronectin. It has been reported to be implicated in several processes such as cell spreading, migration, proliferation, and survival (ZHANG et al., Proceedings of the National Academy of Sciences, 92, 6161-65, 1995; CAO et al., J. Biol. Chem., 273, 31670-79, 1998; MATTER & RUOSLAHTI, J. Biol. Chem., 276, 27757-63, 2001; PULAI et al., Arthritis Rheum, 46, 1528-35, 2002). In the case of osteoblasts, it has been reported that interactions between integrins, including α5β1, α3β1, and α8β1, and extracellular matrix molecules, in particular fibronectin and type I collagen, were involved in the ability of foetal rat calvarial osteoblasts to differentiate into mature osteoblasts able to produce a mineralized extracellular matrix (LYNCH et al., Exp Cell Res, 216, 35-45, 1995; MOURSI et al., J Cell Sci, 109, 1369-80, 1996; MOURSI et al., J Cell Sci, 110, 2187-96, 1997). It has also be shown recently showed that reduction of ITGA5 expression in osteoblasts results in decreased cell adherence and apoptosis in vitro and in vivo (DUFOUR et al., Exp Cell Res, 313, 394-403, 2007; KAABECHE et al., J Cell Sci, 118, 1223-32, 2005). However, no contribution of ITGA5 to osteogenic differentiation of human MSCs has been reported until now.

The inventors have now found that ITGA5 is upregulated during osteoblast differentiation induced by dexamethasone in clonal and primary hMSCs, and that, surprisingly, overexpression or specific activation of ITGA5 is sufficient to induce the commitment of human mesenchymal stem cells towards the osteogenic pathway, and their subsequent differentiation into mature osteoblasts.

Therefore, the present invention proposes the use of an agonist of ITGA5 to promote the differentiation of human mesenchymal stem cells into osteoblasts.

An agonist of ITGA5 is herein defined as a compound which interacts with ITGA5, said interaction resulting in increased intracellular signalling, such as modulation of JNK, PI3K/Akt or ERK1/2 signaling.

Said agonist of ITGA5 can be for instance an antibody specifically directed against ITGA5, such as the anti-α5 monoclonal antibody, that was recently shown to prime α5β1 integrin (CLARK et al., J Cell Sci, 118, 291-300, 2005), or any antibody, like SNAKA51 which has a ligand-induced binding site (LIBS) epitope mapping to the calf domains of the α5-integrin subunit leg region.

Other examples of agonists of ITGA5 are peptides having a high binding affinity for α5β1 integrin (HECKMANN & KESSLER, Methods Enzymol, 426, 463-503, 2007; HUMPHRIES et al., J Cell Sci, 119, 3901-3, 2006; KOIVUNEN et al., J Cell Biol, 124, 373-80, 1994; MOULD et al., J Biol Chem, 273, 25664-72, 1998) and that may act as agonists on ITGA5. These are in particular peptides comprising the sequence RRETAWA (SEQ ID NO: 1).

An object of the present invention is a method for inducing osteoblast differentiation in vitro of human mesenchymal stem cells, wherein said method comprises culturing said human mesenchymal stem cells with an ITGA5 agonist.

Another object of the present invention is the use of an ITGA5 agonist for preparing a medicament for enhancing osteogenesis by promoting osteoblast differentiation of human mesenchymal stem cells.

One application could be to treat autologous mesenchymal stem cells derived from a patient with an ITGA5 agonist to promote osteogenic differentiation before re-implanting these cells to the patient. Another application may be to inject locally at sites of bone loss an ITGA5 agonist to promote bone repair or regeneration. The ITGA5 agonist may also be administered per os alone or chemically associated with a bone seeking agent such as calcium, strontium or a bisphosphonate to target the bone tissue. Additionally, the ITGA5 agonist may be physically immobilized onto osteoconductive biomaterial such as β-tricalcium phosphate, calcium carbonate or natural coral to promote the attachment of mesenchymal stem cells in contact with the implanted biomaterial and thereby promote osteoblast differentiation of these cells and bone repair.

The present invention is useful in the treatment of all diseases associated with bone loss or insufficient bone formation. Such diseases include for instance bone fractures, bone defects, bone resection, osteolysis and bone loss related to endocrine disorders or malignancy.

### EXAMPLE 1

### Analysis of Dexamethasone-Induced Osteoblast Differentiation in hMSCS

Human primary mesenchymal stem cells (MSC) were derived from normal bone marrow stroma as previously described (DELORME & CHARBORD, Methods Mol Med, 140, 67-81, 2007). Briefly, bone marrow cells were obtained from iliac crest aspirates. Nucleated cells were seeded at a density of 5,000 cells/cm² in complete medium supplemented with 1 ng/ml FGF2 (AbCys, Paris, France). Non-adherent cells were removed by changing the medium at day 3; thereafter, medium was changed twice a week.

F/STRO1⁺A cells were derived from human fetal bone marrow stroma, selected for Stro1 antigen expression, immortalized using the large T SV40 and subsequently subcloned (OYAJOBI et al., J Bone Miner Res, 14, 351-61, 1999). This clonal human bone marrow stromal cell line express mRNA markers or protein of the osteoblast lineage (Runx2, OC, ALP, type 1 collagen), of the chondrocyte lineage (aggrecan, types 2, 9 and 10 collagen), and of the adipocyte lineage (PPARgamma2, C/EBPalpha, aP2, G3PDH, LPL, leptin) under basal conditions (AHDJOUDJ et al., J Cell Biochem, 81, 23-38, 2001).

Cells were routinely cultured in Dulbecco's Modified Eagles Medium (DMEM; Invitrogen Corporation, Paisley, Scotland) supplemented with 10% heat inactivated FCS, 1% L-glutamine and penicillin/streptomycin (10,000 U/ml and 10,000 μg/ml, respectively), at 37° C. in humidified atmosphere containing 5% CO₂ in air. Culture media were changed every 2 or 3 days.

For induction of osteoblast differentiation, the cells were treated with dexamethasone at physiological dose (10⁻⁷ M) which is sufficient to promote human MSC osteogenic differentiation Cheng (CHENG et al., Endocrinology, 134, 277-86, 1994; FROMIGUE et al., Cytokine, 9, 613-23, 1997; FROMIGUE et al., J Cell Biochem, 104, 620-8, 2008).

The activity of alkaline phosphatase, an early marker of osteoblast differentiation, was determined by a colorimetric assay as previously described (FROMIGUE et al., J Cell Biochem, 104, 620-8, 2008). ALP staining was performed using Sigma FAST kit according to the manufacturer's recommendations (Sigma). Cells were fixed in 75% ethanol, rinsed in PBS and incubated with the substrate buffer at 37° C.

The expression of mRNA markers of the osteoblast lineage (Runx2, ALP, type 1 collagen) was evaluated by quantitative RT-PCR. Total RNAs were isolated using Trizol reagent (Laboratoires Eurobio, France) according to the manufacturer's instructions. Three μg of total RNA from each sample were reverse transcribed using MMLV reverse transcriptase and oligodT primers, at 37° C. for 90 min. The relative mRNA levels were evaluated by quantitative PCR using LightCycler Instrument (Roche Applied Science, Indianapolis Ind., USA) and SYBR Green PCR kit (ABGen, Courtabceuf, France). Triplicate reactions were carried out for each sample. Signal was normalized to 18S as internal control.

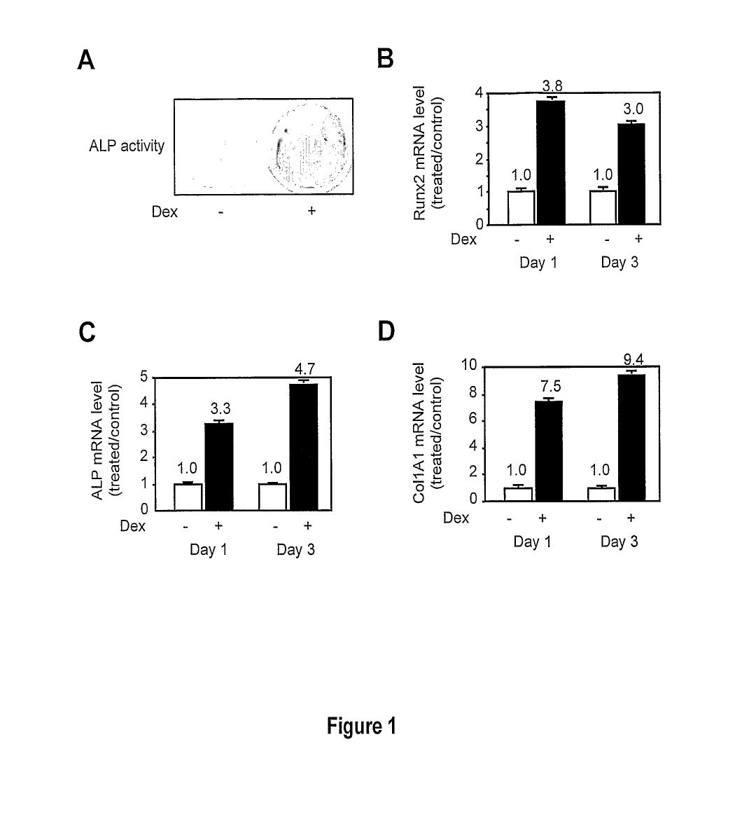

The results obtained in cultures of human primary MSCs are shown in FIG. 1.

Legend: Dexamethasone upregulates osteoblast markers in hMSCs. A) Treatment with dexamethasone (Dex, 10⁻⁷M) increased alkaline phosphatase (ALP) activity in human primary MSCs. B-D) Dex promoted osteoblast genes mRNA levels expression, as evaluated by quantitative RT-PCR. Results, after correction to 18S content, are expressed as mean±SD of treated over control ratio.

As shown in FIG. 1A, dexamethasone at physiological dosage induced a marked increase in ALP activity. Quantitative RT-PCR showed that dexamethasone at the same dosage increased osteoblast markers gene expression (Runx2, ALP, Col1A1) at 1 and 3 days of treatment in hMSCs (FIGS. 1B,C,D). The same results were obtained with the F/STRO1⁺A cell line (results not shown).

These results are consistent with an early induction of osteoblast differentiation by dexamethasone in human primary or clonal MSCs.

We then determined the gene expression profile in primary hMSCs cells that were promoted to differentiate into osteoblasts. Cells were treated with dexamethasone for 1 and 3 days, total RNA was collected, and used for microarray hybridizations.

The microarray analysis was carried out as described previously (DELORME & CHARBORD, Methods Mol Med, 140, 67-81, 2007). Total RNA were extracted using RNeasy kit (Qiagen; Courtaboeuf, France) according to the manufacturer's recommendations, and 5 μg of each samples were submitted to in vitro transcription (ENZO Biochem, New York, N.Y.) to generate biotin-labeled cDNA. Hybridization was carried on HG-U133 Plus 2.0 microarrays, according to standards supplied by the manufacturer (Affymetrix, Santa Clara, Calif.).

Analysis of the normalized microarray data from 3 separate MSCs obtained from different donors revealed that several genes were changed more than 2-fold in dexamethasone-treated MSCs compared to untreated cells. Using the Ingenuity Pathway software database, we identified genes that are involved in the control of cell proliferation or death, cell signaling, or cell differentiation into adipogenesis or other lineages. One of these genes (ITGA5) was found to be upregulated by dexamethasone at day 1 and 3 both in primary MSCs and F/STRO1⁺A cells, suggesting that this gene may play a role in dexamethasone-induced MSC differentiation into osteoblasts.

### EXAMPLE 2

### Up-Regulation of ITGA5 by Dexamethasone in Human MSCs During Osteoblast Differentiation

In order to confirm that ITGA5 expression was effectively up-regulated by dexamethasone in hMSCs during osteoblast differentiation, the expression of ITGA5 mRNA was evaluated by quantitative RT-PCR, using the protocol described in Example 1 and the expression of ITGA5 was evaluated by Western blot analysis.

For Western blot analysis, cells lysates were prepared as previously described (FROMIGUE et al. Cell Death Differ. 13, 1845-56, 2006). Briefly, proteins (30 μg) were resolved on 4-12% SDS-PAGE and electrotransfered onto PVDF nitrocellulose membranes (Millipore Corporation, Bedford, USA). Filters were incubated at RT for 2 h in 50 mM Tris/HCl (pH 7.4), 150 mM NaCl, 0.1% (v/v) Tween-20, 0.5% (w/v) bovine serum albumin (TBST/BSA), then overnight at 4° C. on a shaker with specific primary antibodies diluted at 1/1000 in TBST/BSA: monoclonal antibody against the housekeeping gene GAPDH (Abeam Cambridge, UK), and rabbit polyclonal antibody directed against ITGA5 was Santa Cruz Biotechnology (Santa Cruz, Calif., USA). Membranes were washed twice with TBST and incubated for 2 hours with appropriate HRP-conjugated secondary antibody (1/20,000 in TBST/BSA). After final washes, the signals were visualized with enhanced chemiluminescence western blotting detection reagent (ECL, Amersham Biosciences, Piscataway, N.J., USA) and autoradiographic film (X-OMAT-AR, Eastman Kodak Company, Rochester, N.Y., USA). Densitometric analysis using ImageQuant software was performed following digital scanning (Agfa).

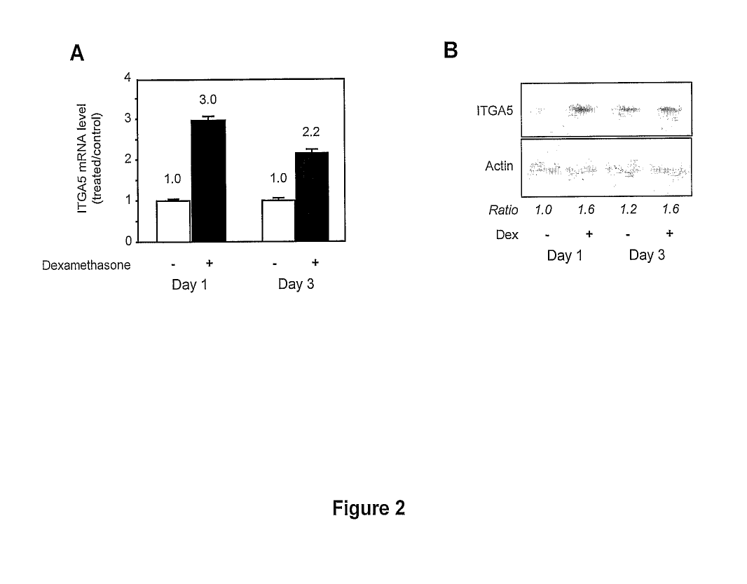

The results are shown in FIG. 2.

Legend: Dexamethasone increases ITGA5 expression in hMSCs. A) Treatment with dexamethasone (Dex, 10⁻⁷ M) increased ITGA5 mRNA level expression, at days 1 and 3, as evaluated by quantitative RT-PCR, in hMSCs. Results, after correction to 18S content, are expressed as mean±SD of treated over control ratio. B) Western blot analysis showing that Dex increased ITGA5 protein levels in hMSCs. Treated over control ratio, after correction to actin content, are mentioned.

As shown in FIG. 2A, quantitative RT-PCR analysis confirmed that dexamethasone greatly increased by 2-3-fold the expression of ITGA5 in primary hMSCs at day 1 and 3, thus validating the microarray analysis.

As shown in FIG. 2B, dexamethasone increased by about 1.5-fold ITGA5 protein levels in hMSCs, further validating the microarray at the protein level.

ITGA5 mRNA levels were also increased in F/STRO1⁺A cells at 1 and 3 days during dexamethasone-induced differentiation (results not shown), confirming the upregulation of this gene during early stages of osteoblast differentiation in MSCs.

We therefore sought to determine the role of ITGA5 in osteoblast differentiation in human MSCs.

### EXAMPLE 3

### Effect of ITGA5 on Osteoblast Differentiation in hMSCs

### Overexpression of ITGA5

To determine the role of overexpression of ITGA5 in mesenchymal cell differentiation, MSCs were stably infected with a lentivirus expressing ITGA5.

### Plasmids Contructs

The human ITGA5 CDS was amplified by PCR from pcDNA3 from Drs S. Kuwada and X. Li (University of Utah, USA) using 5′-CAGGGAAGAGCGGGCGCTATGG-3′ (SEQ ID NO: 2) and 5′-GGGAGTCTGAAATTGGGAGGACTCAGG-3′ (SEQ ID NO: 3) primers. The amplified ITGA5 CDS sequence was cloned into pCR8/GW/TOPO TA plasmid (Invitrogen), then transferred into the pLentiGW plasmid (Invitrogen) by in vitro recombination.

sh-ITGA5 encoding sequence was obtained by PCR elongation of the primers 5′-GGATCCCCGTGACTTCTTTGCCGTGAATTCAAGAGATTCA-3′ (SEQ ID NO: 4) and 5′-AAGCTTAAAAAGTGACTACTTTGCCGTGAATCTCTTGAAT-3′ (SEQ ID NO: 5). This shITGA5 encoding sequence was cloned into pGEMT easy vector (Promega) and transferred into the pH1 plasmid (gift from Anne Galy, Généthon-Evry) in BglII/HindIII sites. The H1 promoter—shITGA5 sequence was cloned into the pLenti-RNAi vector using SpeI and ClaI sites.

### Lentiviral Particles Production and Transduction

Viral production was performed using human embryonic kidney cells HEK293T grown in DMEM supplemented with 10% FBS, 1% L-glutamine and penicillin/streptomycin (10,000 U/ml and 10,000 μg/ml, respectively), and 2 mM Hepes. The day before transfection, 2×10⁶ cells were seeded on 175 cm² flask. Lentiviral transfer vector (LV-ITGAS) (50 μg), VSV-G viral envelope plasmid (ph-CMV-G) (10 μg) and packaging construct (pCMV DR8.74) (50 μg) were mixed with water up to 800 μl and 200 μl of 2 M CaCl₂ and then added to 1 ml of Hepes-buffered saline solution 2×(5M NaCl₂, 1M KCl, 150 mM Nα₂HPO₄, 0.5M Hepes; pH 7) and incubated at RT for 20 min. This DNA solution was then added drop wise onto HEK293T cells with medium, swirled gently and then incubated overnight at 37° C. with 5% CO₂. The following day, the transfection solution was removed, the cells were rinsed with serum-free medium before addition of 15 ml of complete medium. After 24 and 48 hours incubation, the supernatants were collected, centrifuged at 1200 rpm to remove cell debris, and filtered through a 0.45 μm low protein binding filter (Corning, Bath, UK), aliquoted and stored at −80° C.

For transduction, sub-confluent recipient cells were incubated with lentivirus and 4 μg/ml polybrene in complete medium. After 48 hours, transduction medium was discarded and cells were ready for experiments. Under these conditions, the transfection efficiency was >90%, as evaluated by GFP staining.

We then analysed the impact of ITGA5 on hMSC differentiation in these conditions, by analyzing its effects on osteoblast markers, as described in Example 1, and on in vitro osteogenesis.

To assess the impact of ITGA5 on in vitro osteogenesis, MSCs transduced with ITGA5 were cultured for 10 days in a culture medium was supplemented with 50 μM ascorbic acid and 3 mM inorganic phosphate (NaH₂PO₄; Sigma) to induce extracellular matrix mineralization. Cells were fixed in 4% paraformaldehyde in PBS. Matrix mineralization was evaluated by alizarin red staining as previously described (FROMIGUE et al., J Cell Biochem, 104, 620-8, 2008) and microphotographed using an Olympus microsocope.

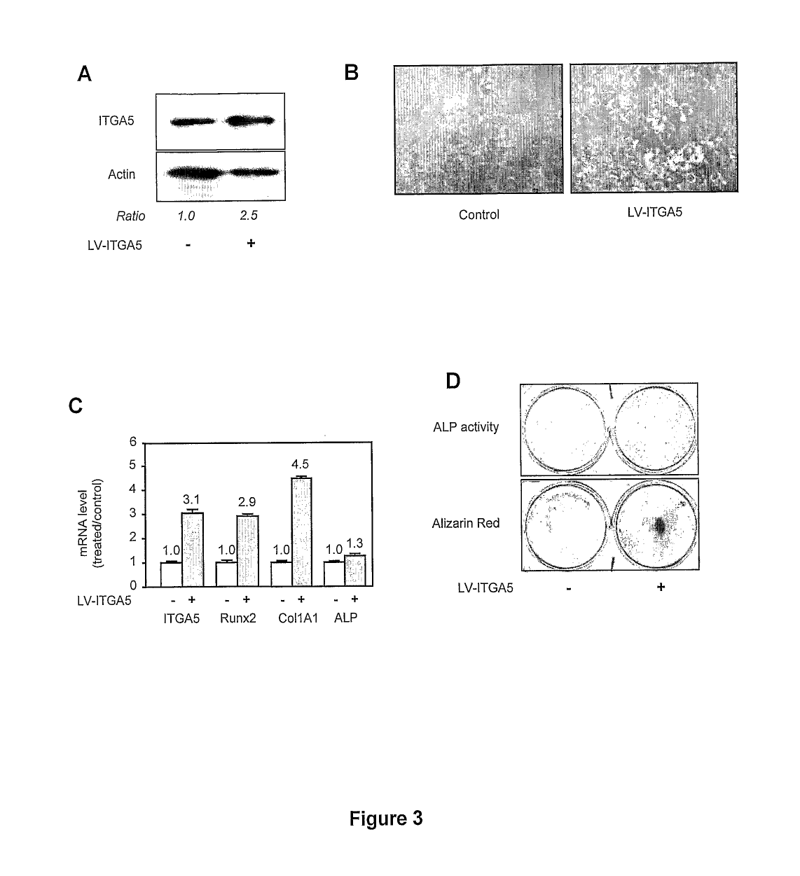

The results are shown in FIG. 3.

Legend: ITGA5 promotes osteogenic differentiation in human MSCs. Over-expression of ITGA5 using lentivirus encoding ITGA5 increased ITGA5 protein level, as shown by western blot analysis (A) and immunocytochemistry (B). ITGA5 over-expression increased ALP, Runx2 and CollA1 mRNA levels, as determined by quantitative RT-PCR, in human MSCs. Results, after correction to 18S content, are expressed as mean±SD of treated over control ratio (C). ITGA5 over-expression increased ALP activity and in vitro matrix mineralization, as revealed by alizarin red staining, in human MSCs (D).

As shown in FIG. 3A, the lentiviral transduction in hMSCs increased ITGA5 protein level expression by 2.5-fold, as determined by western blot analysis. Overexpression of ITGA5 in hMSCs was confirmed by immunocytochemistry (FIG. 3B).

We then analysed the impact of ITGA5 on hMSC differentiation in these conditions. As shown in FIG. 3C, ITGA5 transduction in hMSCs increased ITGA5 (as expected) and Runx2, ALP and Col1 A1 mRNA expression, as determined by quantitative RT-PCR analysis. Similar results were found in three other different hMSCs obtained from different donors (data not shown). Consistent with this effect on osteoblast markers, we found that overexpression of ITGA5 in MSCs increased ALP activity (FIG. 3D). We also confirmed that ITGA5 overexpression increased ALP activity and osteoblast marker genes in clonal F/STRO1⁺A cells (data not shown).

As shown in FIG. 3D, ITGA5 overexpression greatly increased the osteogenic capacity of primary MSCs in vitro. These results suggest that a transient expression of ITGA5 is sufficient to promote phenotypic osteoblast markers and osteogenic capacity in MSCs.

siRNA-Mediated Silencing of ITGA5

To further establish the role of ITGA5 in osteoblast differentiation of MSCs, we determined whether RNA-interference-mediated silencing of ITGA5 expression may interefer with osteoblast differentiation. To this goal, we used murine siRNA and analysed its effect on the basal expression of osteoblast markers.

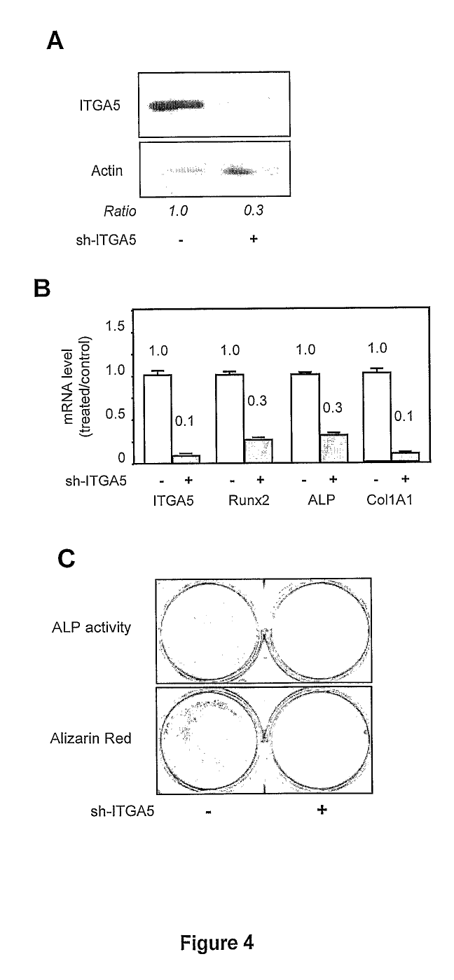

The results are shown in FIG. 4.

Legend: ITGA5 silencing reduces osteoblastic gene expression in hMSCs. A) hMSCs were transduced with a lentiviral vector encoding ITGA5 sh-RNA (sh-ITGA5) or a non relevant sh-RNA (−) and ITGA5 protein level was determined by western blot analysis. Relative expression (after correction for actin) is mentioned. B) ITGA5 silencing using specific sh-RNA reduced ITGA5, Runx2, ALP and Col1A1 mRNA levels evaluated by qRT-PCR in hMSCs compared to a non relevant sh-RNA. Results, after correction to 18S content, are expressed as mean±SD of treated over control ratio. C) sh-ITGA5 transduction reduced ALP activity in hMSCs, as evaluated by histochemical staining, and reduced in vitro matrix mineralization, as revealed by alizarin red staining, compared to a non relevant sh-RNA.

As shown in FIG. 4A, ITGA5 silencing using sh-RNA reduced ITGA5 protein level as determined by western blot analysis. As shown in FIG. 4B, sh-ITGA5 reduced ITGA5, Runx2, ALP and Col1A1 mRNA levels compared to a non relevant sh-RNA. Results, after correction to 18S content, are expressed as treated over control ratio. C) sh-ITGA5 transduction reduced ALP activity, as evaluated by histochemical staining, and reduced in vitro matrix mineralization, as revealed by alizarin red staining, in human MSCs compared to a non relevant sh-RNA.

### EXAMPLE 4

### Activation of ITGA5 is Sufficient to Induce Osteoblast Differentiation in hMSCs

Having established that ITGA5 exerts functional effects on osteoblast differentiation in hMSCs, we sought to determine whether activation of ITGA5 alone may be effective in promoting hMSC differentiation. To this goal, we used a conformation-dependent anti-α5 monoclonal antibody (SNAKA51) that was recently shown to prime α5β1 integrin and promote cell adhesion and ligand-binding in fibroblasts (CLARK et al., J Cell Sci, 118, 291-300, 2005).

hMSCs were incubated with SNAKA51 (provided by Dr. M. J. Humphries, University of Manchester, UK) at the dose of 10 μg/ml for 24 h, RNA were collected and osteoblast markers were determined. To determine the functional impact of increasing ligand-binding on in vitro osteogenesis, MSCs were cultured in the presence of the SNAKA51 monoclonal antibody at the dose of 10 μg/ml for 10 days, in the presence of ascorbic acid and phosphate and in vitro osteogenic capacity was determined.

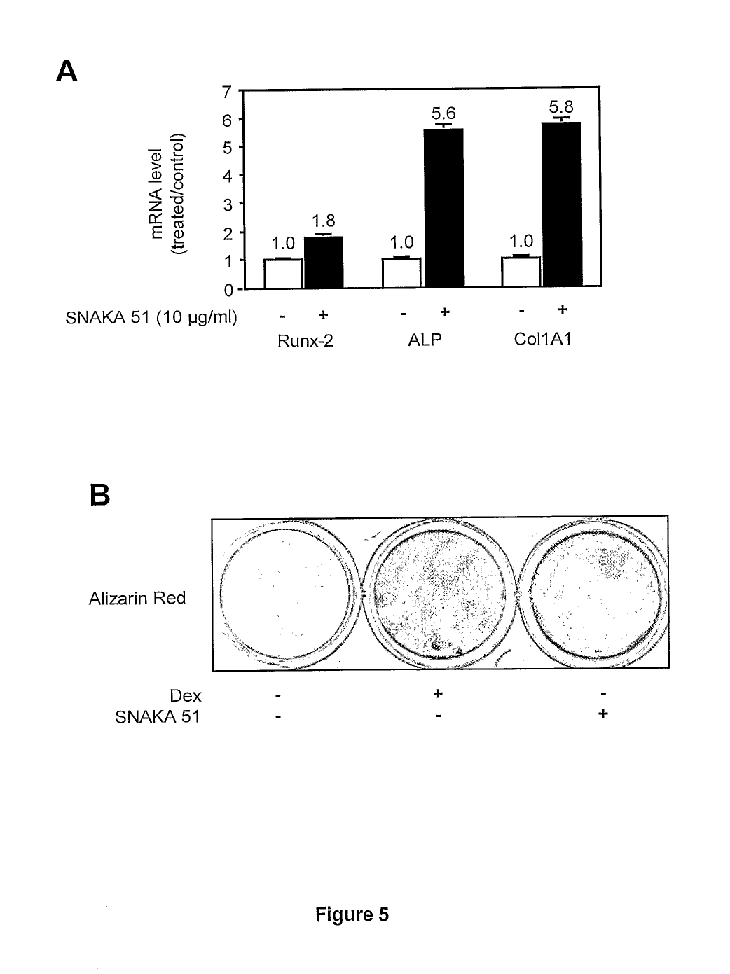

The results are shown in FIG. 5.

Legend: Priming ITGA5 with anti-ITGA5 antibody promotes osteogenic differentiation of hMSCs. A) Treatment of hMSCs with the ITGA5 antibody (SNAKA51) for 24 h increased Runx2, ALP and ColA1 mRNA levels, as determined by quantitative RT-PCR, in human MSCs. Results, after correction to 18S content, are expressed as mean±SD of treated over control ratio. B) Treatment of hMSCs with SNAKA51 increased in vitro matrix mineralization, as revealed by alizarin red staining. Treatment with dexamethasone (Dex, 10⁻⁷ M) is shown for comparison.

We found that the addition of SNAKA51 at a dose that was found to promote ligand-binding in fibroblasts did increase the expression of Runx2, ALP and Col1A1 in primary MSCs (FIG. 5A). Similar results were found in clonal MSCs (F/STRO1⁺A cells) (data not shown). We also show that SNAKA51 greatly increased the osteogenic capacity of primary hMSCs in vitro (FIG. 5B).

The above results provide evidence that activation of ITGA5 using a specific monoclonal antibody that primes the integrin is sufficient to promote phenotypic osteoblast markers and osteogenic capacity in cultured human MSCs.

To confirm the finding that treatment of hMSCs with an ITGA5 agonist promotes osteoblast differentiation, we used a synthetic cyclic peptide (*CRRETAWAC* (SEQ ID NO: 6), provided by Dr. E. Ruoslahti, Cancer Research Center, La Jolla, Calif., USA) that is a specific and selective peptide ligand for α5β1 (KOIVUNEN et al., J Cell Biol, 124,3: 373-80, 1994).

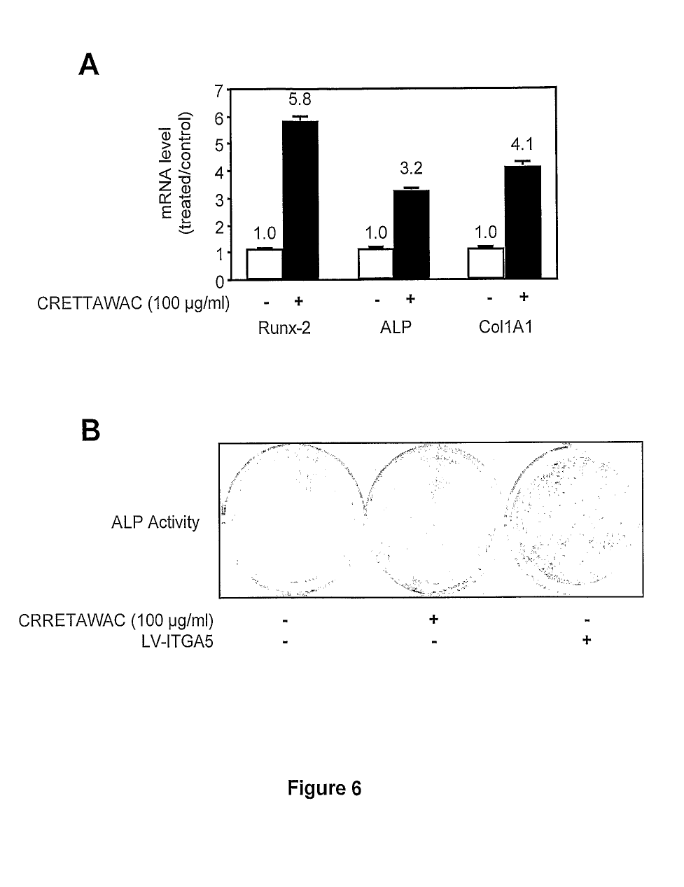

The results are shown in FIG. 6.

Legend: The synthetic cyclic peptide CRRETAWAC promotes osteoblast marker genes expression in hMSCs. A) Human MSCs were treated with CRRETAWAC at the dose of 100 μg/ml for 24 h, RNAs were collected and osteoblast markers were determined by qRT-PCR analysis. Results, after correction to 18S content, are expressed as mean±SD of treated over control ratio. B) Coating with 100 μg/ml of CRRETAWAC increased ALP activity, as revealed by staining, in clonal F/STRO1⁺A cells. Infection with LV-ITGA5 is shown for comparison.

These results show that the peptide CRRETAWAC acting as agonist of ITGA5 greatly increased the expression of osteoblast markers in MSCs in vitro.

The above results provide evidence that activation of ITGA5 using a specific monoclonal antibody or a synthetic cyclic peptide that selectively primes ITGA5 is sufficient to promote phenotypic osteoblast markers and osteogenic capacity in cultured human MSCs.

### EXAMPLE 5

### Osteoblast differentiation induced by ITGA5 in hMSCs Involves ERK Signalling

Having established that ITGA5 activation promotes osteoblast differentiation in hMSCs, we sought to identify the underlying signalling pathway involved in this effect. To this goal, proteins collected from hMSCs infected with LV-ITGA5 were analysed by western blot analysis.

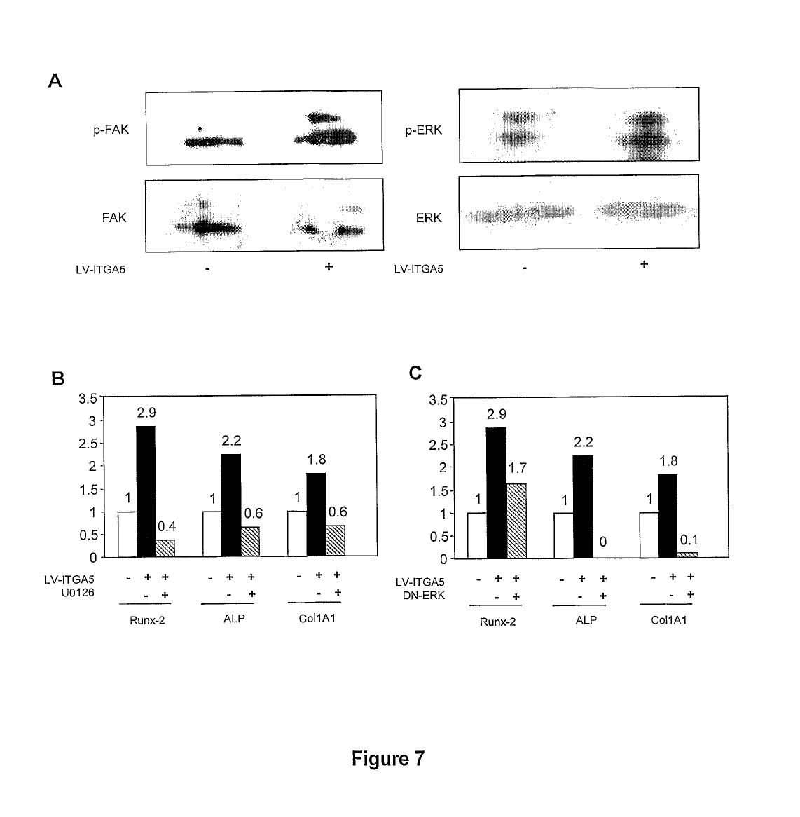

The results are shown in FIG. 7.

Legend: Osteoblast differentiation by ITGA5 involves ERK1/2 signalling. A) ITGA5 overexpression increases Focal Adhesion Kinase (FAK) phosphorylation and downstream Extracellular Related Kinase (ERK) 1/2 phosphorylation. B) Treatment of LV-ITGA5-infected hMSCs with the MEK inhibitor U0126 (10 μM) blunted the increased Runx2, ALP and ColA1 mRNA levels induced by ITGA5 overexpression, as determined by quantitative RT-PCR. C) Transient transfection with DN-ERK (24 hours) reduced the increased Runx2, ALP and ColA1 mRNA levels induced by ITGA5 overexpression, as determined by quantitative RT-PCR. Results, after correction to 18S content, are expressed as treated over control ratio.

To establish the role of ERK1/2 in osteoblast differentiation induced by ITGA5 in hMSCs, we used a selective ERK inhibitor. We found that the addition of U0126, an inhibitor of MAPK kinase 1 and 2 (MEK1/2) that blocks phosphorylation and activation of ERK1/2, blunted the increased expression of Runx2, ALP and Col1A1 induced by ITGA5 overexpression in primary MSCs (FIG. 7B). Similar results were found in clonal MSCs (F/STRO1⁺A cells) (data not shown).

To further confirm the role of ERK1/2 signalling in ITGA5-induced osteoblast differentiation in hMSCs, LV-ITGA5-infected cells were transiently transfected with ERK1/2 dominant-negative (DN-ERK) vector that reduces ERK signalling (PAGES et al., Proc Natl Acad Sci USA. 90, 8319-8323, 1993). As shown in FIG. 7C, DN-ERIC reduced Runx2 and blunted the increased ALP and Col1A1 mRNA expression induced by ITGA5 overexpression in hMSCs. Similar results were found in clonal MSCs (F/STRO1⁺A cells) (data not shown).

The above results indicate that ITGA5-induced activation of osteoblast differentiation markers in cultured human MSCs is mediated, at least in part, through activation of the ERK1/2 signalling pathway.

1. A method for promoting osteoblast differentiation in vitro of mesenchymal stem cells, wherein said method comprises culturing said human mesenchymal stem cells with an agonist of the integrin α5 subunit (ITGA5).

2. The method of claim 1, wherein said agonist of the integrin α5 subunit is selected among:
an anti-ITGA5 antibody recognizing an epitope mapping to the calf domains of the integrin α5 subunit leg region;
a peptide comprising the sequence RRETAWA (SEQ ID NO: 1).

3. A method for enhancing osteogenesis comprising administering an amount of an agonist of an integrin α5 subunit effective for promoting osteoblast differentiation of mesenchymal stem cells.

4. The method of claim 3, wherein said agonist of the integrin α5 subunit is selected among:
an anti-ITGA5 antibody recognizing an epitope mapping to the calf domains of the integrin α5 subunit leg region;
a peptide comprising the sequence RRETAWA (SEQ ID NO: 1).

5. An agonist of the integrin α5 subunit for inducing osteoblast differentiation of mesenchymal stem cells.

6. An agonist of the integrin α5 subunit for inducing osteoblast differentiation of mesenchymal stem cells according to claim 5, wherein said agonist of the integrin α5 subunit is selected among:
an anti-ITGA5 antibody recognizing an epitope mapping to the calf domains of the integrin α5 subunit leg region;
a peptide comprising the sequence RRETAWA (SEQ ID NO: 1).

7. A composition for inducing osteoblast differentiation of mesenchymal stem cells, wherein said composition comprises an agonist of the integrin α5 subunit.

8. The composition of claim 7, wherein said agonist of the integrin α5 subunit is selected among:
an anti-ITGA5 antibody recognizing an epitope mapping to the calf domains of the integrin α5 subunit leg region;
a peptide comprising the sequence RRETAWA (SEQ ID NO: 1).
